(19) United States
(12) Patent Application Publication
(10) Pub. No.: US 2023/0128111 A1
(43) Pub. Date: Apr. 27, 2023

(54) Joint Sparse Estimation for Covariate Selection in Decision Support Causal Modeling

(71) Applicant: International Business Machines Corporation, Armonk, NY (US)

(72) Inventors: Kristjan Herbert Greenewald, Belmont, MA (US); Karthikeyan Shanmugam, Elmsford, NY (US); Dmitriy A. Katz, Ossining, NY (US)

(21) Appl. No.: 17/511,723

(22) Filed: Oct. 27, 2021

(51) Int. Cl. G16H 50/20; G06F 17/18

(52) U.S. Cl. CPC G16H 50/20; G06F 17/18

(57) ABSTRACT

Estimator mechanisms for automated computer causal effect estimation are provided. An input dataset is received that includes an initial set of covariate data. An estimation of the relevance of covariates in the initial set is performed where relevance is to one or more causal effect relationships between a given at least one action and an outcome. Based on results of the execution of the estimation, a subset of the initial set of covariates is determined that are covariates relevant to one or more causal effect relationships. A modified dataset, comprising the subset of relevant covariates and at least a portion of the input dataset is generated. The modified dataset is input to a causal effect estimator that processes the modified dataset to generate causal effect relationship estimates for specifying causal effects between the given set of actions and the outcome.

### STATEMENT REGARDING PRIOR DISCLOSURES BY THE INVENTOR OR A JOINT INVENTOR

The following disclosure(s) are submitted under 35 U.S.C. 102(b)(1)(A):

DISCLOSURE(S): “High-Dimensional Feature Selection for Sample Efficient Treatment Effect Estimation”, Kristjan Greenewald, Dmitriy Katz-Rogozhnikov, Karthik Shanmugam, arXiv:2011.01979v1 [stat.ML], Nov. 3, 2020.

### BACKGROUND

The present application relates generally to an improved computing tool and improved computing operation for performing covariate selection in decision support causal modeling, and more specifically for performing such selection based on a new joint sparsity estimation process.

Decision-support systems exist in many different industries where human experts require assistance in retrieving and analyzing information. An example is a diagnosis system employed in the healthcare industry. Diagnosis systems can be classified into systems that use structured knowledge, systems that use unstructured knowledge, and systems that use clinical decision formulas, rules, trees, or algorithms. The earliest diagnosis systems used structured knowledge or classical, manually constructed knowledge bases. The Internist-I system developed in the 1970s used disease-finding relations and disease-disease relations. The MYCIN system for diagnosing infectious diseases, also developed in the 1970s, used structured knowledge in the form of production rules, stating that if certain facts are true, then one can conclude certain other facts with a given certainty factor. DXplain, developed starting in the 1980s, used structured knowledge similar to that of Internist-I, but adds a hierarchical lexicon of findings. Iliad, developed starting in the 1990s, added more sophisticated probabilistic reasoning where each disease had an associated a priori probability of the disease (in the population for which Iliad was designed), and a list of findings along with the fraction of patients with the disease who have the finding (sensitivity), and the fraction of patients without the disease who have the finding (1-specificity).

In 2000, diagnosis systems using unstructured knowledge started to appear. These systems used some structuring of knowledge such as, for example, entities, e.g., findings and disorders, being tagged in documents to facilitate retrieval. ISABEL, for example, used Autonomy information retrieval software and a database of medical textbooks to retrieve appropriate diagnoses given input findings. Autonomy Auminence used the Autonomy technology to retrieve diagnoses given findings and organized the diagnoses by body system. First CONSULT allowed one to search a large collection of medical books, journals, and guidelines by chief complaints and age group to arrive at possible diagnoses. PEPID DDX provided a diagnosis generator based on PEPID’s independent clinical content.

More recently, health decision making systems, computer based diagnostic systems, and the like, have moved into the realm of advance artificial intelligence (AI) solutions. While the health industry has become very good at accumulating data, much of this data goes untapped and its value is lost due to inaccessibility or poor organization. In fact, it is estimated that healthcare data will experience a compound annual growth rate of 36% through 2025. Only after data is aggregated, secured, and analyzed can its value be realized. Financial resources can be saved by managing resources more effectively and time can be saved by uncovering data trends faster. Most importantly, individuals can benefit when care information is accessible when it is needed most, and organizations can use the data to help guard the health and safety of people they serve.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided, in a data processing system comprising at least one processor and at least one memory, the memory comprising instructions executed by the at least one processor to specifically configure the at least one processor to implement an estimator for automated computer causal effect estimation. The method comprises receiving, by the estimator, the input dataset comprising first data and second data, the second data comprising an initial set of covariate data. The method further comprises executing, by the estimator, an estimation of relevance of covariates, in the initial set of covariate data, to one or more causal effect relationships between at least one action and an outcome. In addition, the method comprises identifying, by the estimator, based on results of the execution of the estimation, a subset of the second data that are covariates relevant to the one or more causal effect relationships, to thereby generate a subset of relevant covariates. The method also comprises generating, by the estimator, a modified dataset comprising the subset of relevant covariates and at least a portion of the first data. Furthermore, the method comprises inputting, by the estimator, the modified dataset to a causal effect estimator that processes the modified dataset to generate causal effect relationship estimates for specifying causal effects between the at least one action and the outcome.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is an example data flow involving a joint sparsity estimator in accordance with one illustrative embodiment;

FIG. 2 is an example causal graph illustrating a general scenario of estimating the treatment effect of a treatment (T) on a univariate outcome (Y) in the presence of covariates (X);

FIG. 3 is an example diagram of a causal model for explaining an overall concept of a joint sparsity estimator operation in accordance with one illustrative embodiment;

FIG. 4 is a flowchart outlining an example operation of a joint sparsity estimator mechanism in accordance with one illustrative embodiment;

FIG. 5 is an example diagram of a distributed data processing system in which aspects of the illustrative embodiments may be implemented; and

FIG. 6 is an example block diagram of a computing device in which aspects of the illustrative embodiments may be implemented.

### DETAILED DESCRIPTION

Decision-making support computing tools operate to assist human beings in making decisions by gleaning insights from observational data through complex computer specific data collection, analysis, and accessibility tools. Such decision-making support computing systems perform operations that are too complex to be practically performed by human beings, either through mental processes, or through organizing of human activity, for a variety of different reasons including the sheer volume of data that must be analyzed, the complexity of the relationships and patterns buried within the voluminous data, the large sets of variables that must be considered, and the like. Because of this complexity, recent decision-making support computing tools have focused on various types of artificial intelligence (AI) solutions, such as machine learning computer models, e.g., neural networks, random forest trees, Support Vector Machines (SVMs), deep learning neural networks, and the like, to be able to identify patterns in large datasets. These patterns can drive predictions and classifications output by these AI computing tools.

In the area of causal effect modeling for assisting with decision making, other types of decision-making support computing tools and AI solutions include doubly robust estimators which combine outcome regression with a model for exposure to an action or condition to estimate the causal effect of the exposure on an outcome. When used individually to estimate a causal effect, both outcome regression and exposure modeling are unbiased only if the statistical model is correctly specified. By combining these two approaches, the doubly robust estimator makes it such that only one of the two models needs to be correctly specified in order to obtain an unbiased effect estimator. It should be appreciated that while the illustrative embodiments will be described with regard to improving the operation of a doubly robust estimator, the illustrative embodiments are not limited to such and may be used with any artificial intelligence or decision-making support computing tool in which there are covariates that need to be evaluated to determine the causal effect of an independent variable on a dependent variable in the computer modeling (see discussion hereafter). Thus, the illustrative embodiments may be implemented with regard to doubly robust estimators, machine learning computer models, or other artificial intelligence computing tools, without departing from the spirit and scope of the present invention.

One area of decision-making support to which AI computing tools, e.g., doubly robust estimators, neural networks, and the like, may be applied is the area of predictions with regard to the effect that actions will have on a desired outcome. That is, by analyzing large datasets with AI computing tools, the AI computing tools may be able to evaluate and predict the effect that various different types of actions can have on a situation so that decision-making can be made as to which actions to perform to achieve a desired outcome. This sort of predictive AI computing operation may be performed with regard to various domains of data. For example, in the healthcare domain, such predictive AI computing operations may be used to evaluate drugs/treatment policies, and public health interventions. In the business domain, such evaluations may be used to evaluate the effect of business decisions, policies, and the like. In economic domains, such predictive AI computing operations may be used to understand the effect of economic policy options, e.g., modifying interest rates, tax policies, and quantitative easing.

In order to accurately estimate causal effects from observational data, it is important to not only have data for the cause and the effect, but also for as many covariates as possible. A covariate is a variable that affects a dependent variable, but is not of interest in the study itself. That is, in statistical analysis of data, there are independent variables (also referred to as predictor variables or explanatory variables) which are variables that explain the variation in a dependent variable (also referred to as a response variable or outcome variable), which is a variable whose value responds to changes in the independent variable. The covariate may affect the dependent variable but is not the primary focus of the particular relationship being studied. For example, suppose a researcher wishes to know, for a set of students, if three different studying techniques lead to different exam scores. In this scenario, the studying technique is the independent variable and the exam score is the dependent variable. However, there are other factors that may affect the dependent variable, e.g., the innate abilities of the students themselves, whether they got sufficient sleep the night before the test, whether they have personal situations that affect their ability to concentrate, and a plethora of other factors that are not specifically the independent variable, i.e., the studying technique. These are the covariates that should be taken into account to improve the accuracy of the cause-effect study.

The evaluation of covariates is used to de-bias the operations performed by the decision-making support computing tools. Real world systems often have very large numbers of covariates. Unfortunately, the more covariates that are observed in the large scale input datasets, which is important for de-biasing, the more data is required for low variance operations. Such considerations are especially true in healthcare decision-making support computing tools, e.g., AI or cognitive computing based decision-making support computing tools which may look to causal relationships to help inform decision making. As such causal thinking spreads from the healthcare domain across other areas of science and business, it is important to ensure that cause effect estimates can be accurately estimated with realistic amounts of data. However, current solutions do not provide sufficient solutions for handling the voluminous amount of data and large numbers of covariates. As a result, feature selection for identifying a subset of relevant covariates within large datasets is either not performed, resulting in too much data being required, or only limited simple feature selection is performed that does not scale as the number of possible actions (independent variables), and thus, the number of covariates, being evaluated increases.

The illustrative embodiments provide an improved computing tool and improved computing operation for performing covariate selection in decision support causal modeling, and more specifically for performing such selection based on a new joint sparsity estimation process performed by a specifically configured improved computing tool, referred to herein as a joint sparsity estimator. The joint sparsity estimator operates based on a joint sparsity objective function, i.e., a function that specifies a system objective as a function of decision variables indicating how much each variable contributes to the system objective, which is optimized to find a small set of relevant covariates that predict each conditional outcome well. That is, given a set of historical observation data for a plurality of possible actions of interest, along with a large set of observed covariates associated with these possible actions, the joint sparsity estimator pares down the large number of observed covariates to a small number of relevant covariates.

After paring down the set of covariates, a causal effect estimator or other AI model may be applied to model the cause and effect relationship between the possible actions and corresponding outcomes. The causal effect estimator uses the smaller relevant set of covariates as input, along with the other input data, rather than the larger set of all covariates, which may include covariates that are not relevant to the cause and effect relationships between possible actions and outcomes. As a result, more accurate, and more resource efficient in terms of computing time, energy, and resource availability, modeling of cause and effect relationships for decision-making support computer modeling is achieved. That is, the cause and effect relationship estimator or AI computing tool modeling of each possible action is achieved more efficiently and accurately by only having to evaluate the input data along with a smaller set of covariates that have been determined to be relevant to the particular cause and effect relationships, rather than a much larger and more computationally complex set of covariates which may not improve the relationship modeling, but will require larger amounts of resources to evaluate.

Before beginning the discussion of the various aspects of the illustrative embodiments and the improved computer operations performed by the illustrative embodiments, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on hardware to thereby configure the hardware to implement the specialized functionality of the present invention which the hardware would not otherwise be able to perform, software instructions stored on a medium such that the instructions are readily executable by hardware to thereby specifically configure the hardware to perform the recited functionality and specific computer operations described herein, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software executing on computer hardware, specialized computer hardware and/or firmware, or any combination thereof that performs the specified functions including, but not limited to, any use of a general and/or specialized processor in combination with appropriate software loaded or stored in a machine readable memory and executed by the processor to thereby specifically configure the processor to perform the specific functions of the illustrative embodiments. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user’s computer, partly on the user’s computer, as a stand-alone software package, partly on the user’s computer and partly on a remote computer or entirely on the remote computer or server. In the latter scenario, the remote computer may be connected to the user’s computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The present invention may be a specifically configured computing system, configured with hardware and/or software that is itself specifically configured to implement the particular mechanisms and functionality described herein, a method implemented by the specifically configured computing system, and/or a computer program product comprising software logic that is loaded into a computing system to specifically configure the computing system to implement the mechanisms and functionality described herein. Whether recited as a system, method, of computer program product, it should be appreciated that the illustrative embodiments described herein are specifically directed to an improved computing tool and the methodology implemented by this improved computing tool. In particular, the improved computing tool of the illustrative embodiments specifically provides improved computer functionality with regard to selection of relevant covariates to include in causal effect estimations by automated computing tools which improves the accuracy and performance of the causal effect estimator computing tools. The improved computing tool implements mechanism and functionality, such as a joint sparsity estimator, which cannot be practically performed by human beings either outside of, or with the assistance of, a technical environment, such as a mental process or the like. The improved computing tool provides a practical application of the methodology at least in that the improved computing tool is able to intelligently identify covariates that are relevant to a causal effect relationship and include in the information processed by causal effect estimators only those covariates that are determined to be relevant to the causal effect relationship.

The illustrative embodiments provide an improved computing tool in the form of a joint sparsity estimator and joint sparsity estimation process that involves the use of a joint sparsity objective function to select a subset of covariates from a larger set of covariates in historically observed data. The selected subset of covariates are those determined, by application of the joint sparsity objective function and optimization of such by the joint sparsity estimator, to be the most relevant to a particular set of possible actions that are to be evaluated with regard to cause and effect on a particular outcome. For example, assume that one wants to determine the effect of smoking on birth weight for newborns. The actions may be no smoking, X cigarettes daily, Y cigarettes daily, and Z cigarettes daily. The outcome may be birth weight of the newborn babies of the individual patients. There may be a large set, e.g., hundreds, of covariates present in an input dataset based on historically observed data for individual patients, e.g., patient weight, patient height, patient medical history variables, e.g., previous pregnancies, previous operations undergone, various medical conditions of the patient, other medications the patient is taking, alcohol consumption, diet, physical activity level, etc., but only a small subset may actually be relevant to the cause and effect relationship of smoking to birth weight. The mechanisms of the illustrative embodiments identify which of those covariates are relevant to the particular cause and effect relationship of the actions and outcome and configures the causal effect model, e.g., the causal effect estimator or AI computer model, so that only the smaller relevant set of covariates need be evaluated along with the input data rather than having to evaluate all of the covariates present.

The following description will first provide an overall description of the improved computing tool and improved computing tool operation with regard to the joint sparsity estimator and joint sparsity estimation process. Thereafter, the basis for the joint sparsity objective function and joint sparsity operations performed by the joint sparsity estimator and joint sparsity estimation process will be described in greater detail. In this description, it will be assumed for purposes of illustration, that the joint sparsity estimator and joint sparsity estimation process are applied to a healthcare domain where the actions evaluated are a particular treatment for a medical condition and the outcomes are the effect of the treatment on the patient with regard to the medical condition being treated. Moreover, it will be assumed that the causal effect estimator is a doubly robust estimator that evaluates each possible action (e.g., treatment of interest) and estimates the outcome using the subset or relevant covariates determined by the joint sparsity estimator. This causal effect estimator may take many different forms depending on the desired implementation. For example, an average treatment effect (ATE) or individual treatment effect (ITE) causal effect estimator may be utilized. While the illustrative embodiments will assume a healthcare domain and a doubly robust estimator are utilized, it will be apparent to those of ordinary skill in the art that the mechanisms of the illustrative embodiments may be implemented with other domains of interest and with other types of estimators and/or AI computer models that operate on a set of possible actions to estimate or predict the potential effects or outcomes of these actions, without departing from the spirit and scope of the present invention.

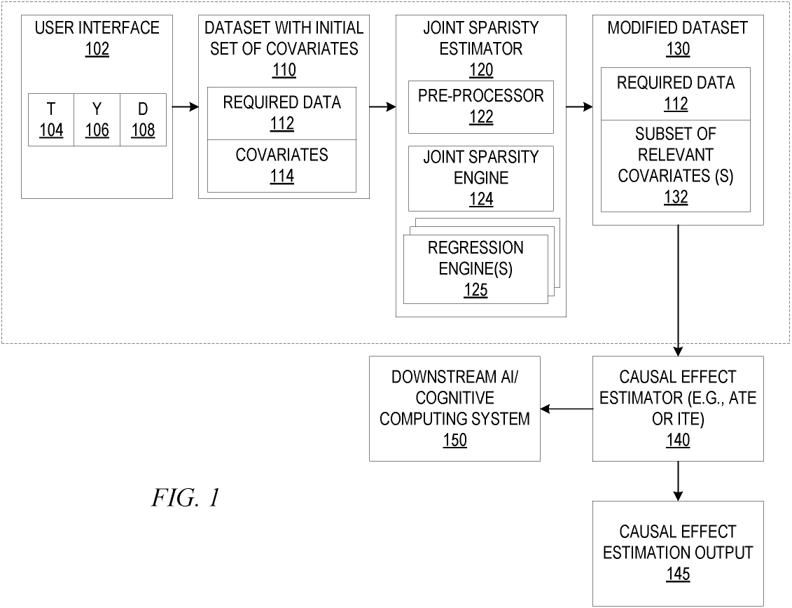

FIG. 1 is an example data flow involving a joint sparsity estimator in accordance with one illustrative embodiment. The elements shown in FIG. 1 are implemented by one or more specifically configured computing devices that are specifically configured to implement artificial intelligence computer models of a joint sparsity estimator 120 and a causal effect estimator 140 that perform operations, that are not practically performed by human beings, to evaluate large volumes of input data and determine a subset of covariates that are of particular relevance to a particular cause and effect relationship being evaluated (joint sparsity estimator 120), and model the cause and effect relationships between specified actions and an outcome based on the subset of relevant covariates (causal effect estimator 140). These operations are automatically performed based on the inputs from a user that specifies the initial set of actions to be evaluated, and the outcome upon which the cause and effect relationship of the actions is to be evaluated. Thus, the operation and data flow outlined in FIG. 1 is intended to represent an automated process but for the initial input and the potential use by human beings of the output for purposes of decision making.

As shown in FIG. 1, the data flow 100 includes a user interface 102 through which a user specifies a list of possible actions of interest that can be taken whose causal effect on a specified outcome of interest is to be modeled through the downstream cause effect estimator 140. That is, assuming a healthcare domain, the user interface 102 provides a graphical user interface (GUI) or other interface through which the user specifies particular actions, such as medical treatments that may be administered, medical conditions of the patient, patient actions that can be performed (e.g., physical exercise, different levels of smoking, taking medications, etc.), levels of care (e.g., high intensity care, medium intensity care, low intensity care), medical procedures performed, or any other input action or condition that can have a causal effect on a specified outcome. The outcomes of interest may be particular medical condition outcomes, for example, such as birth weight, particular clinical values (e.g., blood pressure levels, blood sugar levels, tumor size, etc.), etc. The cause and effect relationships may specify various qualitative or quantitative changes to the outcome due to the action, e.g., increasing/decreasing, improving or not improving, etc.

As noted previously, the illustrative embodiments are not limited to healthcare cause and effect estimators or AI computer models, and may be applied to other domains where cause and effect relationships are to be evaluated through AI computer modeling. For example, in a business domain, a service provider may wish to determine how to keep customers from unsubscribing from their service and may want to know what an account manager can do to keep a customer from canceling their account. In such a situation, the mechanisms of the illustrative embodiments may operate to determine the covariates associated with customers maintaining/canceling their accounts for the service, in association with actions taken by account managers that resulted in customers maintaining/canceling their accounts. The observational data in this case may include many different covariates for different customers, only a subset of which are of actual direct causal relevance to whether or not the customer maintained/canceled their accounts and subscription to the service. The illustrative embodiments provide improved computing tools to identify this subset of covariates and configure the causal effect estimator 140 to operate on the input data and only the subset of covariates that are relevant.

The user input to the user interface 102 specifies a list of possible actions of interest 104 that can be taken for a particular scenario that is to be evaluated by the causal effect estimator 140, e.g., different policies to be implemented, different treatments to be applied, different patient actions that could be taken, etc. This list of possible actions of interest may be a selection from a predefined listing, may be a freeform textual input that can be processed via natural language processing to extract features that are then paired with datasets of interest, or the like. In general, any means of creating a list of possible actions is possible, so long as observational data of each possible action is included in the available dataset (i.e., an observed history of each action being used in the past). For purposes of this description, it is assumed that the actions of interest 104 are treatments (T) for a medical condition, or outcome (Y) 106, which is also specified in the user input. The treatments (T) and outcome (Y) specified in the user input are associated with a user specification 108 of a dataset (D) 108. The dataset (D) 110 specified by the user input 108 contains a series of historical observations of the actions or treatments (T) 104 being used in practice with their associated outcome, along with as many observed covariates (X) as possible. Thus, dataset (D) 110 comprises the historical observation data along with covariates (X). That is the dataset (D) 110, also sometimes referred to generally as observational data, comprises a collection of observed actions taken, resulting observed outcomes, and values of all covariates at the particular time of the observation. The covariates are observed data that is not an action or an outcome.

The dataset (D) 110 is input to the joint sparsity estimator 120 which applies a joint sparsity process involving a joint sparsity objective function being optimized to find a smaller set (S) of relevant covariates that predict each conditional outcome well. As shown in FIG. 1, the joint sparsity estimator 120 comprises a pre-processor 122 that takes the input dataset 110 and extracts the features from the dataset 110 and formats the extracted features for use by the joint-sparsity engine 124. The joint-sparsity engine 124 executes a plurality of regression engines 125 to evaluate q regression operations, each regressing the outcome (Y) on the covariates (X) given one of the possible actions (T). The value of “q” in this context is the number of actions being considered, e.g., the user input to the user interface 110 specifies “q” number of actions to be evaluated, e.g., there are q number of different treatments in the actions (T) 104 whose effect on an outcome (Y) 106 are to be evaluated. These regression operations are combined by the joint-sparsity engine 124 with a non-convex joint-sparsity regularization term (rho-lambda or ρ_{λ}) using a joint-sparsity objective function optimization where this joint-sparsity objective function is of the form:

$$\begin{matrix}\hat{θ} = arg {min}_{{\left‖θ\right‖}_{1 , 2} \leq R} \\ \left\{{\sum }_{j = 1}^{q} \left[\frac{1}{2} {θ}_{: j}^{T} \frac{{X}_{j}^{T} {X}_{j}}{n} {θ}_{: j} - \frac{{y}_{j}^{T} {X}_{j}}{n} {θ}_{: j}\right] + {\sum }_{i = 1}^{p} {p}_{λ} \left({\left‖{θ}_{i :}\right‖}_{2}\right)\right\}\end{matrix}$$

Here, the pairs (Xⱼ,yⱼ) are the portion of the observational data for which action j was taken, and n is the number of data points in the portion. θ is a matrix of size pxq, where jth column (θ_{:j}) of θ is the regression coefficients for predicting the outcome y using X, conditioned on the jth action being taken (j takes values from 1 to q). θ_{i:} is the ith row of θ, and ̂θ̂ indicates the solution of the objective function, i.e. the output of the regression (the “hat” indicates it is an estimate of some true underlying regression coefficients). Computing the solution of this objective function (finding the arg min and forming θ̂) is implemented by a computer performing proximal gradient descent, i.e. proposing a possible θ, computing the gradient of the objective function at that value, incrementing θ by that gradient multiplied by a constant step size, and then sparsifying the result using soft thresholding at the level λ. This procedure is then repeated until convergence, i.e., until the proposed θ stops changing. θ̂ then set to this final value of θ.

The joint-sparsity objective function links q regression operations, each regressing the outcome Y on the covariates X given one of the q possible values of T using a non-convex joint-sparsity regularization term (rho-lambda or ρ_{λ}). In statistical modeling, regression analysis is a set of statistical processes for estimating the relationships between a dependent variable (e.g., the outcome or response variable) and one or more independent variables (e.g., the actions). The most common form of regression analysis is linear regression, in which one finds the line (or a more complex linear combination) that most closely fits the data according to a specific mathematical criterion. For example, the method of ordinary least squares computes the unique line (or hyperplane) that minimizes the sum of squared differences between the true data and that line (or hyperplane). For specific mathematical reasons, this allows the researcher to estimate the conditional expectation (or population average value) of the dependent variable when the independent variables take on a given set of values. Other forms of regression use slightly different procedures to estimate alternative location parameters (e.g., quantile regression or Necessary Condition Analysis) or estimate the conditional expectation across a broader collection of non-linear models (e.g., nonparametric regression). Thus, in the present case, the various regression operations determine the relationships between each of the covariates and the outcome given one of the action inputs in the possible actions T.

In other words, for each of the q values, i.e., each of the different possible actions or treatments, the portion of the input dataset for which that action was taken is isolated and used to create the corresponding portion of the regression objective function. A joint-sparsity regression operation is then performed where for each of the q actions, the covariates are used to predict the outcome. This regression operation involves a j oint-sparsity regularization term (rho-lamda), or joint-sparsity regularizer, which allows for solving the q linked regression operations in a way that encourages jointly sparse solutions, i.e., solutions where each of the regression operations depend only on a small number of relevant covariates shared across the q regression operations. The joint-sparsity regularizer, in some illustrative embodiments, is a non-convex regularizer which provides strong guarantees of estimator accuracy with less data than a traditional convex regularizer, however the joint-sparsity regularizer does not require a non-convex regularizer and a traditional convex regularizer can also be utilized. While a non-convex regularizer is described as being used in the illustrative embodiments to provide strong guarantees, a non-convex regularizer is not required and other types of regularizers may be used without departing from the spirit and scope of the present invention.

The joint sparsity regularizer provides an ability to select the set S of relevant covariates. To achieve this selection, the joint sparsity regularizer introduces a penalty for including covariates in the regression, i.e., it has a cost that scales with the number of nonzero rows in the coefficient matrix θ. Since the regression seeks to minimize the total cost (expressed as the objective function), it will seek regression solutions θ that use as few covariates as possible while still predicting the outcome well. To balance these goals, the solution will seek to include covariates that, conditioned on the observed action, are most directly predictive of the outcome, i.e., in a causal sense they directly impact the outcome instead of impacting some other covariate which then impacts the outcome.

As an example, imagine a scenario where the outcome is the severity of a patient’s viral infection. The action might be a drug or a vaccine. Consider two (out of many) possible covariates: the age of the patient and the cardiovascular fitness of the patient. However, note that “age of the patient” has a strong impact on expected “cardiovascular fitness.” Imagine the outcome (severity of the infection) does not directly depend on age but does depend on cardiovascular fitness. In this setting, a non-sparse regression will use both covariates to predict the outcome (because both covariates are correlated to the outcome) and achieve a low prediction error. A jointly sparse regression will seek to use as few covariates as possible, and will realize that all the predictive power can be achieved using only the covariates on which the outcome directly depends (the “relevant covariates”) which in this case is cardiovascular fitness. This paring down of the number of covariates leads to a more accurate causal effect estimate when the amount of data available is finite. The balancing of the two goals in the joint sparsity regression (predictive power of regression and using as few covariates as possible) is controlled by the parameter λ in the joint sparsity regularization function. In some illustrative embodiments, the joint sparsity estimator 120 of the illustrative embodiments may use several candidate values of λ and select the one for which the number of selected covariates is as small as possible while the resulting regression prediction error is not increased significantly above the regression error achievable when all covariates are used.

The shared non-zero entries in the resulting coefficient estimates, i.e., the entries of θ (the solution of the regression) for the q regression operations then correspond to the desired set S of relevant covariates. In other words, the non-zero entries in the regressed θ_{:j} (these entries are the same for all q values of j due to the joint sparse regression) then correspond to the desired set S of relevant covariates. The basis for the above joint-sparsity objective function and covariate selection using the joint-sparsity objective function will be described in greater detail hereafter.

Thus, the dataset (D) 110, including a set of input data 112 that is evaluated by a downstream causal effect estimator 140, and a set of covariate data 114 which includes data that may or may not be relevant to the cause and effect relationships being evaluated. The joint sparsity estimator 120 operates on this dataset (D) 110 to identify a subset of covariate data S from the covariates (X) 114 in the dataset (D) 110 that is relevant to the cause and effect relationships between actions of interest (T), i.e., those to be evaluated by the downstream causal effect estimator 140, and the outcome of interest (Y). The resulting dataset 130 comprises the set of input data 112 and the subset of covariate data S 132 that comprises only the covariates determined by the joint sparsity estimator 120 to be relevant to the cause and effect relationships between the actions (T) 104 and the outcome (Y) 106. The resulting modified dataset 130 is input to the downstream causal effect estimator 140 which then performs cause and effect estimation operations to model the cause and effect relationships between the actions (T) 104 and the outcome (Y) 106. The causal effect estimator 140 may be implemented using any desired causal effect estimator, such as an average treatment effects (ATE) estimator, an individual treatment effects (ITE) estimator, or the like. The ATE estimator may operate to evaluate the effect of actions averaged over all the values of covariates that are contained in the dataset 130. The ITE estimator may operate to evaluate the effect of the actions for a specific configuration of the covariates in the dataset 130.

The causal effect estimator 140 may provide outputs indicating cause and effect results for specified actions, e.g., treatments, on an outcome, e.g., patient medical condition, which may then be output to a user, such as via the user interface 102 or other associated user interface of a computing device, such as a health dashboard or the like. Moreover, in some illustrative embodiments, the cause and effect results output by the causal effect estimator 140 may be output to other downstream AI computer models and/or cognitive computing systems 150 that may utilize the output from the causal effect estimator 140 as an input which is used as a basis for performing their further AI and/or cognitive computing system operations, e.g., treatment recommendation operations, medical diagnosis operations, or the like. For example, the causal effect output 145 generated by the causal effect estimator 140 may be used by the AI computing system/cognitive computing system 150 to apply the causal effects to patient electronic medical records (EMRs) of interest to determine what treatments are most likely to result in positive outcomes for the patients. In other illustrative embodiments, the AI computing system/cognitive computing system 150 may operate on such causal effect output 145 to predict costs, generate recommendations, or the like, for various domains.

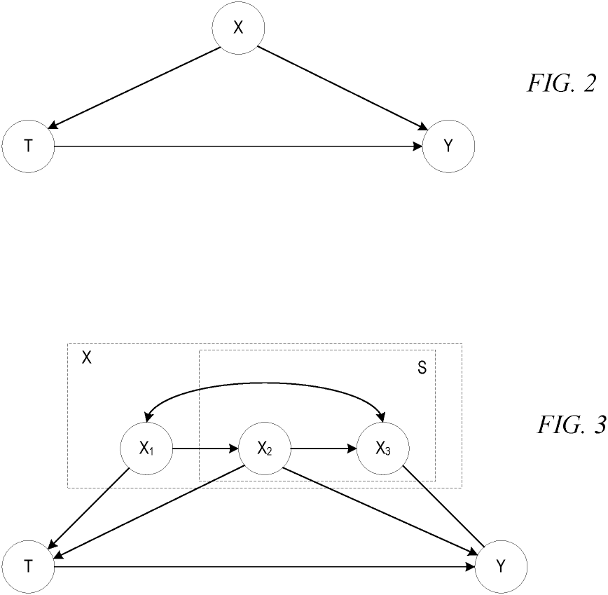

To further illustrate the basis for the joint sparsity estimator 120 operation, consider a causal graph such as shown in FIG. 2. FIG. 2 is an example causal graph illustrating a general scenario of estimating the effect of a treatment (T) on a univariate outcome (Y) in the presence of (possibly confounding) covariates (X), where the treatment variable can take q possible treatment configurations. That is, T is a discrete treatment taking up to q values and Y is a scalar outcome. X is an observed set ofp covariates.

It is assumed that historical observational data is available for the various treatments q which specifies the effects of the various treatments on the patient outcome Y. Denote a “confounder” as any covariate that is causally related to the treatment and, independently of this, to the outcome (e.g., disease status), but is not an intermediate factor on the causal path between the treatment and the outcome. If confounders are not included in the dataset input to the causal effect estimator, the resulting causal effect estimate will be biased. Hence it is important to ensure that the set of covariates includes all potential confounders. Since the set of confounders is not known a priori, in practice as many possible covariates X are included in the dataset. If the set X contains all confounders so that a causal effect estimator using X will yield an unbiased causal effect estimate, the set X is referred to as “admissible.” At the most basic level, X will be “admissible” if given any of the q treatments, the counterfactual outcome associated with that treatment is statistically independent of the treatment choice in the observational data D conditioned on the covariates X.

Consider the simple case when the observed set of covariates X is admissible, i.e., eligible to be used for unbiased effect estimation using the observational data D. Inverse propensity weighing, standardization and doubly robust estimation are techniques that may be used to estimate the causal effect. If the number of covariates included in the set X is large enough to be comparable to the size of the dataset D, the causal effect estimator, using the entire set X, will output a causal effect estimate that has variance too large to be practical in many applications. The illustrative embodiments solve this issue, allowing for low-variance causal effect estimates even when the number of covariates in X is large. This is accomplished by finding a small subset S of X which is also admissible, i.e., it is sufficient to input S to the causal effect estimator to achieve an unbiased causal effect estimate.

To illustrate the overall concept of how a joint sparsity estimator in accordance with the illustrative embodiments recovers this desired subset S, consider a coarse causal model such as depicted in FIG. 3, where the data D, comprising a set of covariates X, has been decomposed into the sets X1, X2, and X3 based on their connections to the outcome Y and treatment T. As can be seen in FIG. 3, X1 either is unconnected to T and Y or has connections to the actions (or treatments) T but not the outcome (Y), X2 has connections to both the actions (T) and the outcome (Y), and X3 has connections to the outcome (Y) but not the actions (T). The identity of these sets are not known a priori and must be discovered from the data D. It can be shown by a simple derivation that in this example, the union of the sets X2 and X3, i.e., those covariates that have connections to either the outcome or both the outcome and the action, constitute a subset S that is admissible. In fact, this is the subset that achieves the lowest variance causal effect estimate. In many practical cases, this subset S or relevant covariates will be much smaller than the set X, as desired.

Previous work focused on finding and controlling for the union of the confounders in the dataset X, i.e., X₁∪X₂, however the illustrative embodiments provide a joint sparsity estimator 120 for the alternative admissible set S with the joint sparsity estimator 120 providing lower sample complexity when the set of treatments T is discrete and the set of outcomes Y continuous. Given historical observational samples of X, Y, and T in the input dataset D 110, the joint sparsity estimator 120 of the illustrative embodiments finds the smallest subset S containing all nodes in X that have an edge pointing towards Y in the causal graph, such as the causal graph shown in FIG. 3. Since it is assumed that the outcome Y does not have any edge pointing to X or T, it is sufficient to use historical observational data to condition on T = t and find the set of nodes Sₜ in X that have edges connecting to Y in the undirected graph, and then take the union over t as

$$\text{S} = {∪}_{t = 1}^{q} {S}_{t} .$$

Group sparsity is encouraged via the L-1,2 norm, which is an L1 norm of the L2 norms of the rows of θ. Thus, the group sparsity based objective can be represented as:

$$\begin{matrix}\hat{θ} = arg {min}_{θ ∈ {ℝ}^{p x q}} {\sum }_{j = 1}^{q} \left[\frac{1}{2} {θ}_{: j}^{T} \frac{{X}_{j}^{T} {X}_{j}}{n} {θ}_{: j} - \frac{{y}_{j}^{T} {X}_{j}}{n} {θ}_{: j}\right] + \\ λ {\left‖θ\right‖}_{1 , 2}\end{matrix}$$

This can be solved iteratively as it is a convex problem, and 2-norm error bounds exist. Unfortunately, it is known that L1 based regression, while successful in estimating coefficients in terms of L2 norm error, does not perform well for variable selection without complex incoherence assumptions. To avoid these difficult to interpret assumptions, instead of L1, the illustrative embodiments rely on the following class of non-convex regularizers that retain the sparsity-promoting properties of the “cusp” at t = 0, while using a nonconvex shape to not excessively penalize large coefficients.

A regularization function ρ_{λ} with parameter λ is µ-amenable for some µ > 0 if the following hold:
ρ_{λ} is symmetric around 0 and ρ_{λ}(0) = 0.
ρ_{λ} is nondecreasing on ℝ+.
the function ρ_{λ}(t)/t is nonincreasing on ℝ+.
ρ_{λ}(t) is differentiable at all t≠0.

$$ρ λ + \frac{μ}{2} {\text{t}}^{2}$$
is convex.
lim_{t→0+} ρ′_{λ}(t) = λ.

If in addition there is some scalar γ ∈ (0, ∞) such that ρ′_{λ}=0 for all t≥ γλ, then ρ_{λ} is (µ, γ)-amenable.

Two example (µ, y) amenable regularizers are the SCAD and MCP penalties. For convenience, define q_{λ}(t) = λ|t| - ρ_{λ}(t). If ρ_{λ} is (µ,γ) amenable, then q_{λ} is everywhere differentiable. Applying a (µ, γ) regularizer ρ_{λ} on the row 2-norms, the following objective function is obtained:

$$\begin{matrix}\hat{θ} = arg {min}_{{\left‖θ\right‖}_{1 , 2} \leq R} \\ \left\{{\sum }_{j = 1}^{q} \left[\frac{1}{2} {θ}_{: j}^{T} \frac{{X}_{j}^{T} {X}_{j}}{n} {θ}_{: j} - \frac{{y}_{j}^{T} {X}_{j}}{n} {θ}_{: j}\right] + {\sum }_{i = 1}^{p} p λ \left({\left‖{θ}_{i :}\right‖}_{2}\right)\right\}\end{matrix}$$

Thus, the joint sparsity estimator 120 uses sparse regression to reduce the sample complexity for estimating causal effects in the presence of large numbers of covariates. The joint sparsity estimator 120 operates based on joint-sparsity promoting non-convex regularization which correctly recovers the sparse support S with high probability. The subset S of relevant covariates found through the optimization of the objective function of the joint sparsity estimator 120 are used to modify the dataset D 110 to generate a modified dataset 130 having a smaller subset of covariates S than the original set of covariates in the dataset D 110. The modified dataset 130 is provided as input to a causal effect estimator 140 and/or other downstream AI computer models or cognitive computing systems for use in performing causal effect relationship analysis and for performing decision-making support operations.

For example, in some illustrative embodiments, the mechanisms of the joint sparsity estimator 120 were utilized to assist with identifying relevant covariates in the evaluation of cause and effect relationships between smoking and birth weight. In this example, the dataset studies the effect of maternal smoking on birth weight of their infants in grams, and consists of 4642 singleton births in Pennsylvania, United States. Actions were defined as 0: no smoking (3778 samples), 1: 15 cigarettes daily (200 samples), 2: 6-10 cigarettes daily (337 samples), and 3: 11 or more cigarettes daily (327 samples). 20 covariates were included in the dataset. Results comparing non-sparse double robust effect estimates and the effect estimates obtained by using the double robust (DR) estimator on a sparse set S of relevant covariates are shown in Table 1.

TABLE 1

Estimated Average Treatment Effect on Smoking/Birth Weight Dataset.

| | | |
|---|---|---|
| | Non-sparse DR Estimate | Sparse DR Estimate |
| Effect of 1 vs. 0 | -151.4 g (21.3) | -195.0 g (28.6) |
| Effect of 2 vs. 0 | -161.9 g (16.6) | -264.0 g (34.2) |
| Effect of 3 vs. 0 | -189.2 g (21.1) | -236.0 g (26.6) |
| Binary Effect (>0 vs. 0) | -162.4 g (8.5) | -239.3 g (10.8) |

A random split of the data was performed to have 20% of the dataset used for the subset S estimation and 80% used for the effect estimation. The sparse approach was tuned via cross validation and on average yields a sparse S of cardinality 10.9 (out of |X|=20). Note that the sparse approach, unlike the full approach, yields a binary effect estimate consistent within the known empirical estimated interval. Specifically, for the last row in Table 1, the true treatment effect is known to be between -250 g and -200 g.

Thus, the Sparse DR Estimate fits this ground truth while the Non-sparse DR Estimate does not, indicating better performance by the mechanisms of the illustrative embodiments. That is, the mechanisms of the illustrative embodiments provide a better match to the ground truth. In other words, the available dataset size is fixed, but too small for the non-sparse DR estimator to find a good estimate while using all covariates. The sparse DR estimator has succeed in finding a small set of relevant covariates, so that the fixed dataset size is now large enough to handle the smaller number of covariates, and no additional bias has been created, which would happen if relevant covariates were removed erroneously.

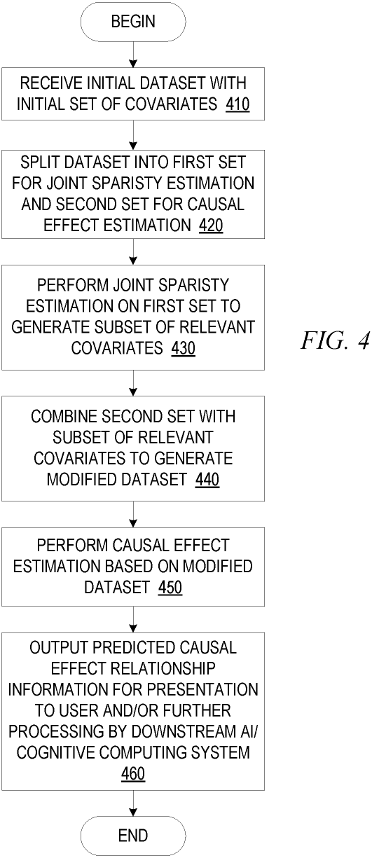

FIG. 4 is a flowchart outlining an example operation of a joint sparsity estimator mechanism in accordance with one illustrative embodiment. The operation outlined in FIG. 4 may be implemented, for example, by a joint sparsity estimator such as element 120 in FIG. 1, for example. The operation may be implemented by software executed on computing hardware to specifically configure the computing hardware to perform the improved computing functions described herein for identifying a subset of covariates that are relevant to a causal effect operation and then configure a causal effect estimator or other AI computing model and/or cognitive computing system to specifically operate on such a modified input dataset having a subset of relevant covariates. The joint sparsity estimator operates to optimize a joint sparsity objective function to thereby identify the subset of relevant covariates.

It should be appreciated that the operations outlined in FIG. 4 are specifically performed automatically by an improved computer tool of the illustrative embodiments and are not intended to be, and cannot practically be, performed by human beings either as mental processes or by organizing human activity. To the contrary, while human beings may initiate the performance of the operation set forth in FIG. 4 and may make use of the results generated as a consequence of the operations set forth in FIG. 4, the operations in FIG. 4 themselves are specifically performed by the improved computing tool in an automated manner.

As shown in FIG. 4, the operation starts with receiving of an initial dataset corresponding to user input specifying the actions and outcome to be evaluated for causal effects (step 410). As noted above, this user input may be received via a user interface and computing system with the user also designating the initial dataset comprising historical observational data for the specified actions and outcome. The dataset preferably includes as many observed covariates as possible for the historical observation data. The dataset is split into a first subset of data used for joint sparsity estimation of a subset of relevant covariates, and a second subset of data for performing the causal effect estimation (step 420). The first subset of data are input to the joint sparsity estimator which executes a joint sparsity engine and regression models on the first subset of data to generate a subset of relevant covariates (step 430). The joint sparsity engine combines the regression operation results, from the regression models for each of the actions being evaluated, using a joint sparsity regularizer (rho-lamda or ρ_{λ}) to generate coefficient estimates, where the non-zero entries in the coefficient estimates for the regression operations correspond to the desired set of relevant covariates.

The second subset of data is combined with the subset of relevant covariates to generate a modified dataset 130 (step 440). The modified dataset is input to a causal effect estimator that processes the second subset of data along with the pared down subset of relevant covariates to generate estimates or predictions of causal effects of the various actions on the outcome (step 450). The predicted or estimated causal effect relationships may then be output or otherwise provided to other downstream AI computing systems and/or cognitive computing systems for performance of artificial intelligence or cognitive computing operations (step 460). The operation then terminates.

From the above, it is clear that the illustrative embodiments provide an improved computing tool and improved computing tool operations that improves the functionality of causal effect estimator computer models and the AI computing systems and/or cognitive computing systems that perform operations based on the predicted or estimated causal effect relationships. The illustrative embodiments provide a joint sparsity estimator that is able to identify which of the many covariates are actually relevant to the causal effect relationships between the specified actions and outcome(s). The joint sparsity estimator effectively reduces the amount of the covariates that must be evaluated to perform causal effect estimation or prediction and thus, reduces resource expenditure to perform such causal effect estimation.

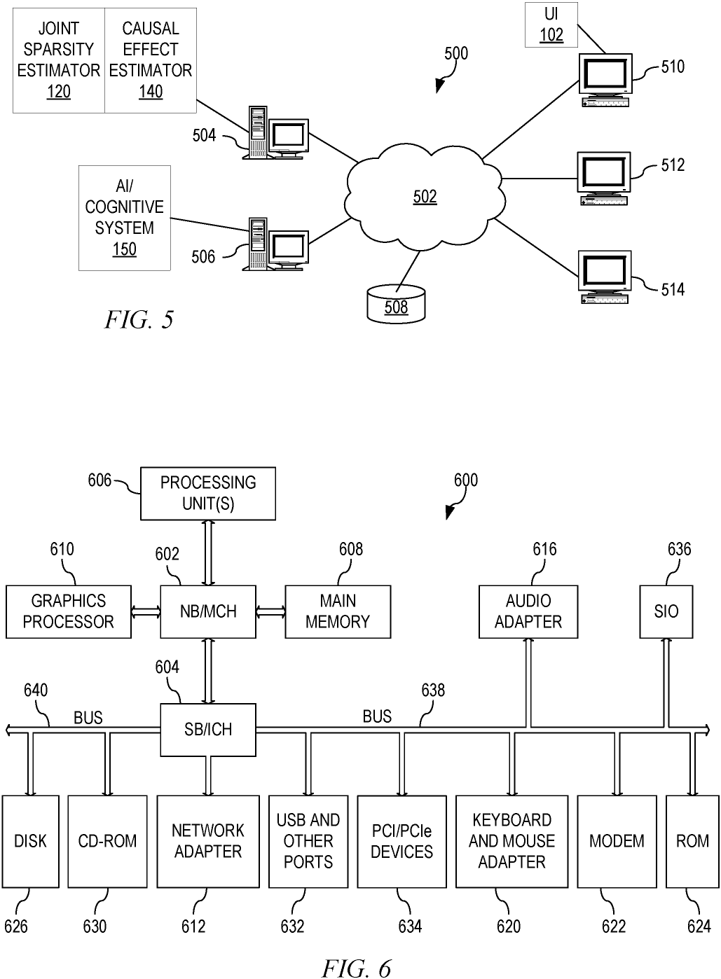

As the illustrative embodiments are directed specifically to an improved computing tool and improved computing tool functionality, the illustrative embodiments may be utilized in many different types of data processing environments. In order to provide a context for the description of the specific elements and functionality of the illustrative embodiments, FIGS. 5 and 2 are provided hereafter as example environments in which aspects of the illustrative embodiments may be implemented. It should be appreciated that FIGS. 5 and 2 are only examples and are not intended to assert or imply any limitation with regard to the environments in which aspects or embodiments of the present invention may be implemented. Many modifications to the depicted environments may be made without departing from the spirit and scope of the present invention.

FIG. 5 depicts a pictorial representation of an example distributed data processing system in which aspects of the illustrative embodiments may be implemented. Distributed data processing system 500 may include a network of computers in which aspects of the illustrative embodiments may be implemented. The distributed data processing system 500 contains at least one network 502, which is the medium used to provide communication links between various devices and computers connected together within distributed data processing system 500. The network 502 may include connections, such as wire, wireless communication links, or fiber optic cables.

In the depicted example, server 504 and server 506 are connected to network 502 along with storage unit 508. In addition, clients 510, 512, and 514 are also connected to network 502. These clients 510, 512, and 514 may be, for example, personal computers, network computers, or the like. In the depicted example, server 504 provides data, such as boot files, operating system images, and applications to the clients 510, 512, and 514. Clients 510, 512, and 514 are clients to server 504 in the depicted example. Distributed data processing system 500 may include additional servers, clients, and other devices not shown.

In the depicted example, distributed data processing system 500 is the Internet with network 502 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers, consisting of thousands of commercial, governmental, educational and other computer systems that route data and messages. Of course, the distributed data processing system 500 may also be implemented to include a number of different types of networks, such as for example, an intranet, a local area network (LAN), a wide area network (WAN), or the like. As stated above, FIG. 5 is intended as an example, not as an architectural limitation for different embodiments of the present invention, and therefore, the particular elements shown in FIG. 5 should not be considered limiting with regard to the environments in which the illustrative embodiments of the present invention may be implemented.

As shown in FIG. 5, one or more of the computing devices, e.g., server 504, may be specifically configured to implement one or more of a joint sparsity estimator (e.g., joint sparsity estimator 120 in FIG. 1), a causal effect estimator (e.g., causal effect estimator 140 in FIG. 1), or an AI computing system/cognitive computing system (e.g., AI/cognitive computer system 150 in FIG. 1) that operates on causal effect relationship estimates. The configuring of the computing device may comprise the providing of application specific hardware, firmware, or the like to facilitate the performance of the operations and generation of the outputs described herein with regard to the illustrative embodiments. The configuring of the computing device may also, or alternatively, comprise the providing of software applications stored in one or more storage devices and loaded into memory of a computing device, such as server 504, for causing one or more hardware processors of the computing device to execute the software applications that configure the processors to perform the operations and generate the outputs described herein with regard to the illustrative embodiments. Moreover, any combination of application specific hardware, firmware, software applications executed on hardware, or the like, may be used without departing from the spirit and scope of the illustrative embodiments.

It should be appreciated that once the computing device is configured in one of these ways, the computing device becomes a specialized computing device specifically configured to implement the mechanisms of the illustrative embodiments and is not a general purpose computing device. Moreover, as described hereafter, the implementation of the mechanisms of the illustrative embodiments improves the functionality of the computing device and provides a useful and concrete result that facilitates relevant covariate identification and filtering out of non-relevant covariates that are not relevant to the particular causal effect relationships of interest. The resulting pared down dataset, comprising required data and a subset of covariates that are relevant to the causal effect relationships, are provided as a basis for causal effect estimation which improves the performance of the causal effect estimators as the causal effect estimators need only evaluate a substantially smaller dataset that includes only those covariates that are determined to be relevant to the causal effect relationships of concern.

In operation, a user of a client computing device, e.g., client computing device 510, may utilize a user interface provided by initiating a session via the data network with the server 504, for example, and inputting a specification of the actions and outcome to be evaluated with regard to causal effect relationships. Moreover, the user may specify the dataset(s) to be used as a basis for performing the causal effect relationship estimation. The dataset(s) may comprise historical observation data for the actions and the outcome, and have an initial set of covariates present in the data, which preferably is as large a set of covariates as possible. The joint sparsity estimator 120 processes the dataset(s) using the joint sparsity estimation model implementing the joint sparsity objective function, to identify the subset of relevant covariates for the particular actions and outcome causal effect relationships. The joint sparsity estimator 120 generates a modified dataset comprising required data and the subset of relevant covariates. The modified dataset is input to the causal effect estimator 140 which estimates the causal effect of each action on the outcome. This causal effect information may then be returned to the client computing device 510 for output to the user for review, or may be provided to another AI computing system/cognitive computing system 150, which may be on the same or a different computing device, for further processing to perform decision making support artificial intelligence operations.

As noted above, the mechanisms of the illustrative embodiments utilize specifically configured computing devices, or data processing systems, to perform the operations for joint sparsity estimation and identification of a subset of relevant covariates in datasets. These computing devices, or data processing systems, may comprise various hardware elements which are specifically configured, either through hardware configuration, software configuration, or a combination of hardware and software configuration, to implement one or more of the systems/subsystems described herein. FIG. 6 is a block diagram of just one example data processing system in which aspects of the illustrative embodiments may be implemented. Data processing system 600 is an example of a computer, such as server 504 in FIG. 5, in which computer usable code or instructions implementing the processes and aspects of the illustrative embodiments of the present invention may be located and/or executed so as to achieve the operation, output, and external effects of the illustrative embodiments as described herein.

In the depicted example, data processing system 600 employs a hub architecture including north bridge and memory controller hub (NB/MCH) 602 and south bridge and input/output (I/O) controller hub (SB/ICH) 604. Processing unit 606, main memory 608, and graphics processor 610 are connected to NB/MCH 602. Graphics processor 610 may be connected to NB/MCH 602 through an accelerated graphics port (AGP).

In the depicted example, local area network (LAN) adapter 612 connects to SB/ICH 604. Audio adapter 616, keyboard and mouse adapter 620, modem 622, read only memory (ROM) 624, hard disk drive (HDD) 626, CD-ROM drive 630, universal serial bus (USB) ports and other communication ports 632, and PCI/PCIe devices 634 connect to SB/ICH 604 through bus 638 and bus 640. PCI/PCIe devices may include, for example, Ethernet adapters, add-in cards, and PC cards for notebook computers. PCI uses a card bus controller, while PCIe does not. ROM 624 may be, for example, a flash basic input/output system (BIOS).

HDD 626 and CD-ROM drive 630 connect to SB/ICH 604 through bus 640. HDD 626 and CD-ROM drive 630 may use, for example, an integrated drive electronics (IDE) or serial advanced technology attachment (SATA) interface. Super I/O (SIO) device 636 may be connected to SB/ICH 604.

An operating system runs on processing unit 606. The operating system coordinates and provides control of various components within the data processing system 600 in FIG. 6. As a client, the operating system may be a commercially available operating system such as Microsoft® Windows 10®. An object-oriented programming system, such as the Java™ programming system, may run in conjunction with the operating system and provides calls to the operating system from Java™ programs or applications executing on data processing system 600.

As a server, data processing system 600 may be, for example, an IBM eServer™ System p® computer system, Power™ processor based computer system, or the like, running the Advanced Interactive Executive (AIX®) operating system or the LINUX® operating system. Data processing system 600 may be a symmetric multiprocessor (SMP) system including a plurality of processors in processing unit 606. Alternatively, a single processor system may be employed.

Instructions for the operating system, the object-oriented programming system, and applications or programs are located on storage devices, such as HDD 626, and may be loaded into main memory 608 for execution by processing unit 606. The processes for illustrative embodiments of the present invention may be performed by processing unit 606 using computer usable program code, which may be located in a memory such as, for example, main memory 608, ROM 624, or in one or more peripheral devices 626 and 630, for example.

A bus system, such as bus 638 or bus 640 as shown in FIG. 6, may be comprised of one or more buses. Of course, the bus system may be implemented using any type of communication fabric or architecture that provides for a transfer of data between different components or devices attached to the fabric or architecture. A communication unit, such as modem 622 or network adapter 612 of FIG. 6, may include one or more devices used to transmit and receive data. A memory may be, for example, main memory 608, ROM 624, or a cache such as found in NB/MCH 602 in FIG. 6.

As mentioned above, in some illustrative embodiments the mechanisms of the illustrative embodiments may be implemented as application specific hardware, firmware, or the like, application software stored in a storage device, such as HDD 626 and loaded into memory, such as main memory 608, for executed by one or more hardware processors, such as processing unit 606, or the like. As such, the computing device shown in FIG. 6 becomes specifically configured to implement the mechanisms of the illustrative embodiments and specifically configured to perform the operations and generate the outputs described herein with regard to the joint sparsity estimator.

Those of ordinary skill in the art will appreciate that the hardware in FIGS. 5 and 6 may vary depending on the implementation. Other internal hardware or peripheral devices, such as flash memory, equivalent non-volatile memory, or optical disk drives and the like, may be used in addition to or in place of the hardware depicted in FIGS. 5 and 6. Also, the processes of the illustrative embodiments may be applied to a multiprocessor data processing system, other than the SMP system mentioned previously, without departing from the spirit and scope of the present invention.

Moreover, the data processing system 600 may take the form of any of a number of different data processing systems including client computing devices, server computing devices, a tablet computer, laptop computer, telephone or other communication device, a personal digital assistant (PDA), or the like. In some illustrative examples, data processing system 600 may be a portable computing device that is configured with flash memory to provide non-volatile memory for storing operating system files and/or user-generated data, for example. Essentially, data processing system 600 may be any known or later developed data processing system without architectural limitation.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising at least one processor and at least one memory, the memory comprising instructions executed by the at least one processor to specifically configure the at least one processor to implement an estimator for automated computer causal effect estimation, the method comprising:
receiving, by the estimator, the input dataset comprising first data and second data, the second data comprising an initial set of covariate data;
executing, by the estimator, an estimation of relevance of covariates, in the initial set of covariate data, to one or more causal effect relationships between at least one action and an outcome;
identifying, by the estimator, based on results of the execution of the estimation, a subset of the second data that are covariates relevant to the one or more causal effect relationships, to thereby generate a subset of relevant covariates;
generating, by the estimator, a modified dataset comprising the subset of relevant covariates and at least a portion of the first data; and
inputting, by the estimator, the modified dataset to a causal effect estimator that processes the modified dataset to generate causal effect relationship estimates for specifying causal effects between the at least one action and the outcome.

2. The method of claim 1, wherein the estimator is a joint sparsity estimator comprising a plurality of regression engines, each regression engine executing a regression operation on a portion of the input dataset specifying a corresponding action in the at least one action, to regress the outcome on covariates, in the initial set of covariate data, given the corresponding action in the at least one action.

3. The method of claim 2, wherein executing the estimation further comprises combining results of the plurality of regression operations by the plurality of regression engines using a joint sparsity objective function and a non-convex joint sparsity regularizer.

4. The method of claim 1, wherein the subset of relevant covariates is smaller in size than the initial set of covariates.

5. The method of claim 1, further comprising:
splitting the first data of the input dataset into a first portion and a second portion, and wherein the first portion is processed by the estimator to generate the subset of relevant covariates, and the second portion is used to generate the modified dataset, wherein the at least a portion of the first data in the modified dataset comprises the second portion of the first data.

6. The method of claim 1, wherein the estimation of relevance of covariates comprises applying a joint sparsity objective function to the input dataset and optimizing the joint sparsity objective function to identify the subset of relevant covariates, wherein the joint sparsity regularizer operates to penalize large subsets of relevant covariates in the optimization of the joint sparsity objective function and to find a minimum size subset of relevant covariates.

7. The method of claim 6, wherein the joint sparsity objective function is defined as:
$$\hat{0} = arg {min}_{{\left‖θ\right‖}_{1 , 2} \leq R} \{ {\sum }_{j = 1}^{q} \left[\frac{1}{2} {θ}_{: j}^{T} \frac{{X}_{j}^{T} {X}_{j}}{n} {θ}_{: j} - \frac{{y}_{j}^{T} {X}_{j}}{n} {θ}_{: j}\right] + \left({\sum }_{i = 1}^{p} {p}_{λ} \left({\left‖{θ}_{i :}\right‖}_{2}\right)\right\}$$
where the pairs (Xⱼ, Yⱼ) are the portion of the observational data X and outcome y for which action j that was taken as indicated in the input dataset, n is the number of data points in the portion of the input dataset corresponding to the action j, θ is a matrix of size pxq, where the jth column (θ_{:j}) of θ is the regression coefficients for predicting the outcome y using X, conditioned on the jth action being taken, θ_{i:} is the ith row of θ, and θ̂ indicates the solution of the objective function.

8. The method of claim 1, wherein the at least one action comprises one or more treatments for a medical condition that may be used to treat the medical condition, wherein the outcome is an effect of the corresponding treatment on a state of the medical condition, and wherein the subset of relevant covariates comprise covariates in the initial set of covariate data that are determined to have a connection to the outcome.

9. The method of claim 8, wherein the causal effect estimator is one of an average treatment effect (ATE) causal effect estimator or an individual treatment effect (ITE) causal effect estimator.

10. The method of claim 1, further comprising inputting the causal effect relationship estimates into a downstream artificial intelligence or cognitive computing system that performs decision making support artificial intelligence operations based on the causal effect relationship estimates.

11. A computer program product comprising a computer readable storage medium having a computer readable program stored therein, wherein the computer readable program, when executed on a computing device, causes the computing device to be specifically configured to implement an estimator for automated computer causal effect estimation, and to:
receive, by the estimator, the input dataset comprising first data and second data, the second data comprising an initial set of covariate data;
execute, by the estimator, an estimation of relevance of covariates, in the initial set of covariate data, to one or more causal effect relationships between at least one action and an outcome;
identify, by the estimator, based on results of the execution of the estimation, a subset of the second data that are covariates relevant to the one or more causal effect relationships, to thereby generate a subset of relevant covariates;
generate, by the estimator, a modified dataset comprising the subset of relevant covariates and at least a portion of the first data; and
input, by the estimator, the modified dataset to a causal effect estimator that processes the modified dataset to generate causal effect relationship estimates for specifying causal effects between the at least one action and the outcome.

12. The computer program product of claim 11, wherein the estimator is a joint sparsity estimator comprising a plurality of regression engines, each regression engine executing a regression operation on a portion of the input dataset specifying a corresponding action in the at least one action, to regress the outcome on covariates, in the initial set of covariate data, given the corresponding action in the at least one action.

13. The computer program product of claim 12, wherein executing the estimation further comprises combining results of the plurality of regression operations by the plurality of regression engines using a joint sparsity objective function and a non-convex joint sparsity regularizer.

14. The computer program product of claim 11, wherein the subset of relevant covariates is smaller in size than the initial set of covariates.

15. The computer program product of claim 11, further comprising:
splitting the first data of the input dataset into a first portion and a second portion, and wherein the first portion is processed by the estimator to generate the subset of relevant covariates, and the second portion is used to generate the modified dataset, wherein the at least a portion of the first data in the modified dataset comprises the second portion of the first data.

16. The computer program product of claim 11, wherein the estimation of relevance of covariates comprises applying a joint sparsity objective function to the input dataset and optimizing the joint sparsity objective function to identify the subset of relevant covariates, wherein the joint sparsity regularizer operates to penalize large subsets of relevant covariates in the optimization of the joint sparsity objective function and to find a minimum size subset of relevant covariates.

17. The computer program product of claim 16, wherein the joint sparsity objective function is defined as:
$$\hat{0} = arg {min}_{{\left‖θ\right‖}_{1 , 2} \leq R} \{ {\sum }_{j = 1}^{q} \left[\frac{1}{2} {θ}_{: j}^{T} \frac{{X}_{j}^{T} {X}_{j}}{n} {θ}_{: j} - \frac{{y}_{j}^{T} {X}_{j}}{n} {θ}_{: j}\right] + \left({\sum }_{i = 1}^{p} {p}_{λ} \left({\left‖{θ}_{i :}\right‖}_{2}\right)\right\}$$
where the pairs (Xⱼ, Yⱼ) are the portion of the observational data X and outcome y for which action j that was taken as indicated in the input dataset, n is the number of data points in the portion of the input dataset corresponding to the action j, θ is a matrix of size pxq, where the jth column (θ_{:j}) of θ is the regression coefficients for predicting the outcome y using X, conditioned on the jth action being taken, θ_{i:} is the ith row of θ, and θ̂ indicates the solution of the objective function.

18. The computer program product of claim 11, wherein the at least one action comprises one or more treatments for a medical condition that may be used to treat the medical condition, wherein the outcome is an effect of the corresponding treatment on a state of the medical condition, and wherein the subset of relevant covariates comprise covariates in the initial set of covariate data that are determined to have a connection to the outcome.

19. The computer program product of claim 18, wherein the causal effect estimator is one of an average treatment effect (ATE) causal effect estimator or an individual treatment effect (ITE) causal effect estimator.

20. An apparatus comprising:
a processor; and
a memory coupled to the processor, wherein the memory comprises instructions which, when executed by the processor, cause the processor to be specifically configured to implement an estimator for automated computer causal effect estimation, and to:
receive, by the estimator, the input dataset comprising first data and second data, the second data comprising an initial set of covariate data;
execute, by the estimator, an estimation of relevance of covariates, in the initial set of covariate data, to one or more causal effect relationships between at least one action and an outcome;
identify, by the estimator, based on results of the execution of the estimation, a subset of the second data that are covariates relevant to the one or more causal effect relationships, to thereby generate a subset of relevant covariates;
generate, by the estimator, a modified dataset comprising the subset of relevant covariates and at least a portion of the first data; and
input, by the estimator, the modified dataset to a causal effect estimator that processes the modified dataset to generate causal effect relationship estimates for specifying causal effects between the at least one action and the outcome.
